(19) United States
(12) Patent Application Publication
(10) Pub. No.: US 2022/0244168 A1
(43) Pub. Date: Aug. 4, 2022

(54) PHOTOTHERMAL GAS DETECTOR INCLUDING AN INTEGRATED ON-CHIP OPTICAL WAVEGUIDE

(71) Applicant: ams AG, Premstaetten (AT)

(72) Inventors: Jochen Kraft, Eindhoven (NL); Rainer Minixhofer, Eindhoven (NL); Victor Sidorov, Eindhoven (NL); Anderson Singulani, Eindhoven (NL); Martin Sagmeister, Eindhoven (NL); Fernando Castano, Eindhoven (NL)

(21) Appl. No.: 17/617,750

(22) PCT Filed: Jul. 9, 2020
(86) PCT No.: PCT/EP2020/069437

§ 371 Date: Dec. 9, 2021

Related U.S. Application Data
(60) Provisional application No. 62/872,362, filed on Jul. 10, 2019.

(51) Int. Cl. G01N 21/17; G01N 33/00

(52) U.S. Cl. CPC G01N 21/171; G01N 33/0027

(57) ABSTRACT

An apparatus includes an integrated waveguide structure, and a first light source operable to produce a probe beam having a first wavelength, wherein the probe beam is coupled into a first end of the waveguide structure. A second light source is operable to produce an excitation beam with having a second wavelength to excite gas molecules in close proximity to a path of the probe beam. A light detector is coupled to a second end of the integrated waveguide structure and is operable to detect the probe beam after it passes through the waveguide structure. The apparatus is operable such that excitation of the gas molecules results in a temperature increase of the gas molecules that induces a change in the probe beam that is measurable by the light detector.

### FIELD OF THE DISCLOSURE

The present disclosure relates to on-chip gas detection systems.

### BACKGROUND

Non-intrusive techniques for detecting low concentrations of trace gases can be useful in a range of environmental, biological and medical applications. Photo-thermal or photo-deflection techniques, for example, are based on the deflection of a light beam by a local refractive index gradient created by the absorption of another light beam by the trace gas.

### SUMMARY

The present disclosure describes a system for the detection of gases based on the photo thermal effect in which the excitation of gas molecules takes place by one light beam (i.e., the pump or excitation beam) having a characteristic wavelength, and where the measurement is performed by another light beam (i.e., the probe beam).

For example, in one aspect, the disclosure describes an apparatus including an integrated waveguide structure. The apparatus further includes a first light source operable to produce a probe beam having a first wavelength, wherein the probe beam is coupled into a first end of the waveguide structure. A second light source is operable to produce an excitation beam having a second wavelength to excite gas molecules in close proximity to a path of the probe beam. The apparatus includes a light detector coupled to a second end of the integrated waveguide structure and operable to detect the probe beam after it passes through the waveguide structure. The apparatus is operable such that excitation of the gas molecules results in a temperature increase of the gas molecules that induces a change in the probe beam that is measurable by the light detector.

Some implementations include one or more of the following features. For example, in some instances, the integrated waveguide structure includes a strip waveguide or a rib wave guide. In some case, the integrated waveguide structure includes at least one of a Fabry-Perot interferometer, a photonic crystal, or a Mach Zehnder interferometer.

In some implementations, the integrated waveguide structure has a reference arm and a probe arm. The apparatus can have at least one opening in a substrate on which the integrated waveguide structure is disposed, such that the at least one opening enables flow of a gas at a location where the excitation beam intersects the probe beam. In some instances, the apparatus has multiple openings in the substrate, wherein the apparatus is operable such that a measurement portion of the probe beam travels through a first one of the openings, and a reference portion of the probe beam travels through a second one of the openings.

In some implementations, the apparatus has an electronic or optical feedback system to control or to tune the excitation beam.

In some cases, a path of the excitation beam intersects the path of the probe beam. Thus, in some implementations, the integrated waveguide structure includes a temperature sensitive part where a path of the excitation beam intersects the path of the probe beam, and wherein a change in temperature of the temperature sensitive part induces a change in the probe beam that is measurable by the light detector. The apparatus can be arranged such that a path of the excitation beam follows in close proximity to the path of the probe beam through the integrated waveguide structure. In some cases, the path of the excitation beam passes a portion of the integrated waveguide structure. A path of the excitation beam may intersect the path of the probe beam during free space propagation of the probe beam.

Depending on the implementation, the second light source can be operable in a pulsed mode or in a continuous mode. The apparatus can include an optical element operable to direct the excitation beam toward an area where the excitation and probe beams intersect. In some implementations, the apparatus includes a light guide to guide the excitation beam from the second light source to a grating coupler, wherein the grating coupler is operable to direct the excitation beam toward an area where the excitation and probe beams intersect.

Although the first and second wavelengths may be the same as one another, in some instances, the excitation beam has a wavelength different from the wavelength of the probe beam.

In another aspect, the disclosure describes a method that includes producing a probe beam having a first wavelength, and coupling the probe beam into a first end of an integrated waveguide structure. The method further includes producing an excitation beam having a second wavelength to excite gas molecules in close proximity to a path of the probe beam, wherein excitation of the gas molecules results in a temperature increase of the gas molecules that induces a change of phase and/or intensity in the probe beam. A light detector coupled to a second end of the integrated waveguide structure is used to measure the change in the probe beam.

Depending on the application, the system can be used to recognize the presence of the gas molecules, to identify the particular gas molecule type, and/or to determine the gas concentration based on the detector output signals. Using an integrated optical waveguide can help make the system more compact, more sensitive, and/or less costly to manufacture in some cases.

Other aspects, features and advantages will be apparent from the following detailed description, the accompanying drawings and the claims.

### BRIEF DSECRIPTION OF THE DRAWINGS

FIG. 1 is a schematic cross-sectional view of an example of a photo-thermal gas detection system.

FIG. 2 illustrates further details of the photo-thermal gas detection system according to some implementations.

FIG. 3 is a schematic cross-sectional view of another example of a photo-thermal gas detection system.

FIG. 4 is a schematic cross-sectional view of a further example of a photo-thermal gas detection system.

FIG. 5 is a schematic view of an example of a photo-thermal gas detection system including a Mach-Zehnder interferometer.

FIG. 6 is a schematic view of another example of a photo-thermal gas detection system including a Mach-Zehnder interferometer.

FIGS. 7A and 7B illustrate further details of the system of FIG. 6 according to some implementations.

### DETAILED DESCRIPTION

The present disclosure describes a system for the detection of gases based on the photo-thermal effect in which the excitation of gas molecules takes place by a first light beam (i.e., the pump or excitation beam) having a characteristic wavelength, and where the measurement is performed by a second light beam (i.e., the probe beam) having a different wavelength. The photo-thermal detection technique relies on deflection of the probe beam when it travels through a medium having a refractive index gradient perpendicular to the propagation direction of the beam. The refractive index gradient is induced by the excitation beam. Absorption of the excitation beam by the gas molecules results in a local increase in temperature, which in turn leads to a temperature gradient and thus a change in refractive index. Deflection of the probe beam is indicative of the amount of excitation light absorbed. Therefore, the probe deflection is proportional to the density of the gas molecules absorbing the excitation light.

As described in greater detail below, the photo-thermal gas detection system can include an integrated on-chip optical waveguide that helps guide the probe beam through one or more parts of the system. Using an integrated optical waveguide can help make the system more compact, more sensitive, and/or less costly to manufacture in some cases.

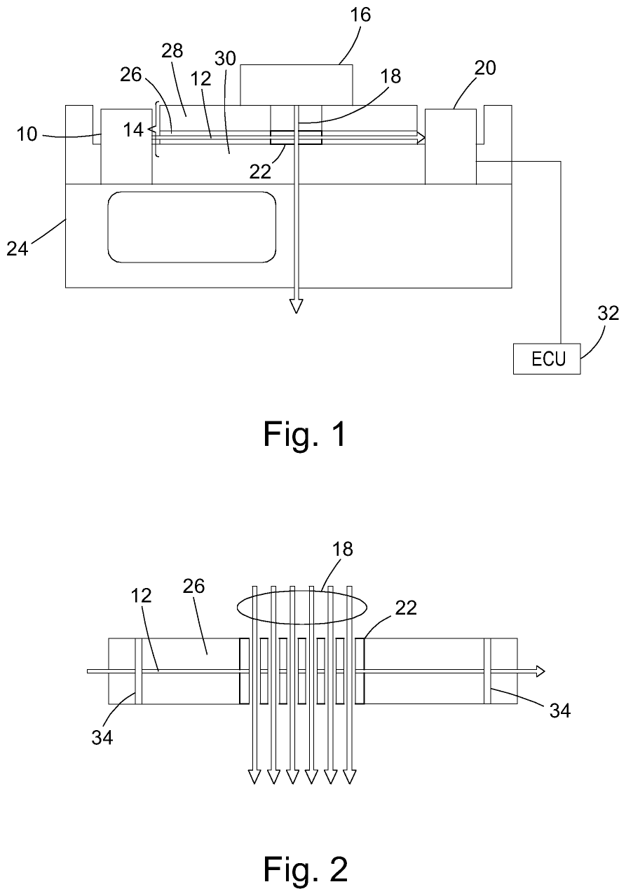

As illustrated in the example of FIG. 1, a system includes a first light source 10 (e.g., a laser device) operable to produce a probe beam 12, which is fed into an integrated optical waveguide structure 14. The waveguide structure 14, which can be implemented, for example, as a slab waveguide having a core 26 surrounded by respective cladding layers 28, 30, can be formed on a silicon or other substrate 24. The relative refractive indices of the core 26 and the claddings 28, 30 are chosen so that the probe beam 12 is guided through the core region by total internal reflection (i.e., the refractive index of the core 26 is larger than that of the surrounding layers 28, 30). Other types of integrated waveguide structures including strip waveguides, rib waveguides, and photonic crystal waveguides, can be used in some instances.

The system also includes second light source 16 operable to produce a pump beam 18, having a wavelength that coincides with a strong characteristic absorption line of a target gas molecule type. In some instances, the second light source 16 is tunable so as to produce light beams having different respective wavelengths. Using a tunable light source allows for the testing of the presence of gas molecule types having different respective absorption lines (e.g., infra-red (IR), etc.). In some implementations, the second light source 16 is a VCSEL or other laser device operable to produce a pump beam having a narrow bandwidth, and having a central wavelength that coincides with a strong absorption line of a particular gas molecule type. The VCSEL or other laser device can be tunable in a wavelength range around this absorption line.

The system further includes a light detector 20 for sensing the probe beam 18 after it passes through the waveguide structure 14. Thus, the waveguide structure 14 is arranged to receive the probe beam 12 at one end, and to direct the probe beam 12 to the light detector 20 as the probe beam exits the waveguide structure 14. In the example of FIG. 1, the path of the pump beam 18 is substantially perpendicular to the path of the probe beam 12 and to the waveguide structure 14. Thus, in this case, the pump beam 18 crosses the waveguide structure 14 and intersects the probe beam 12.

As illustrated in FIG. 1, the first light source 10 and the light detector 20 can be mounted on the substrate 24. In some cases, the light detector may be formed in or on the substrate. For example, if the substrate 24 is composed of silicon, and the first light source 10 is operable to produce visible light for the probe beam 12, then the light detector 20 may be implemented as a photodiode formed, at least in part, in the substrate 24.

In the example of FIG. 1, at least a part 22 of the waveguide structure 14 is sensitive to a change in temperature. For example, a change in temperature may produce a shift in the refractive index of the temperature sensitive part 22 or may result in a thermal extension caused by a longer path length. When the targeted gas molecules are adjacent to, or in contact with, the surface of the temperature sensitive part 22 of the waveguide structure 14, the gas molecules heat up as the pump beam 18 passes through the waveguide structure. As a result of the increased temperature of the temperature sensitive part 22 and the associated shift, e.g., in its refractive index, the evanescent field of the probe beam 12 traveling through the waveguide structure 14 may be influenced. Thus, the amplitude and/or phase of the probe beam 12 may be affected, and can be measured by the light sensor 20. For example, in some instances, as the probe beam 12 passes through the gas volume to be analyzed, the detector 20 detects an intensity change when the gas molecules are present, because of the RI change in the sensitive intensity. The detector only needs to be sensitive in the wavelength range of the probe beam. Thus, for example, even if the absorption line is in the infra red region, the detector can be in the range of visible light, which can make production of the detector much easier and less expensive. Output signals from the detector 20 can be provided to an electronic control unit (ECU) 32 configured to recognize the presence of the gas molecules, to identify the particular gas molecule type, and to determine the gas concentration based on the detector output signals. Preferably, the ECU 32 is an integrated part of the substrate 24, which can make production of the device less expensive.

In some implementations, the temperature sensitive part 22 of the waveguide structure 14 can be realized, for example, as a photonic crystal. In such cases, the interaction between the investigated gas molecules and the waveguide may be stronger as the gas molecules can penetrate through the holes of the photonic crystal into the waveguide. Further, a slow light concept can be realized using a photonic crystal, which can enable an enhanced interaction of the light with the medium having the changed refractive index.

As illustrated in FIG. 2, respective vertical mirrors 34 can be disposed on both sides of the temperature sensitive part 22 of the waveguide structure 14 to form a Fabry-Perot interferometer. The steep transfer characteristic of a Fabry-Perot interferometer can result in the device being very sensitive to small wavelength changes.

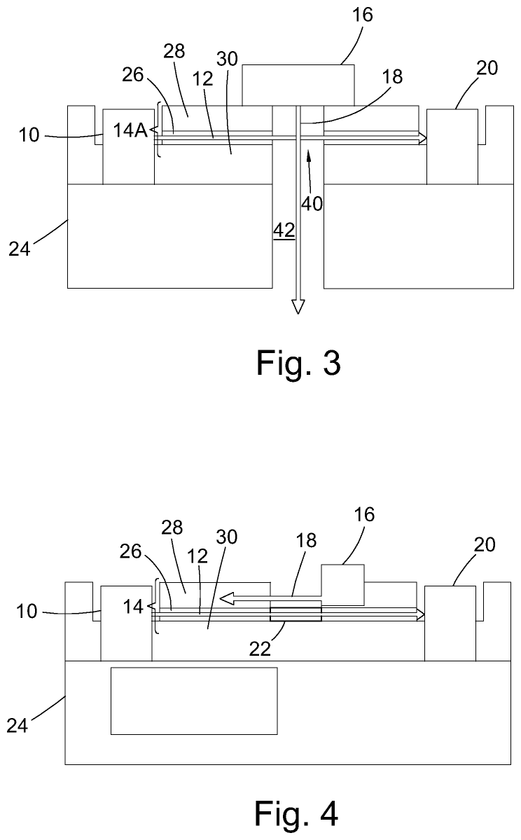

As shown in FIG. 3, in some implementations, instead of incorporating a temperature sensitive element into the waveguide structure, the waveguide structure 14A has a free space region 40 in the area where the pump beam 18 and the probe beam 12 intersect. In such cases, free space propagation of the probe beam 12 enables the direct heating of the medium through which the probe beam is propagating. Thus, indirect heating (i.e., of a temperature sensitive element) is unnecessary. This approach can be advantageous because the through-hole 42 in the substrate 24 enables a gas to flow through the probe beam 12, and the pump beam 18 heats up the gas molecules through which the probe beam 12 passes. In this case, the refractive index of the air in the free space region 40 changes as a result of the gas molecules heating up. The change in refractive index affects the amplitude and/or phase, which can be measured by the light detector 20.

In the foregoing examples, the pump beam 18 travels along a path substantially perpendicular to the probe beam 12. In other implementations, the light sourcel6 can be arranged such that the pump beam 18 travels along a path substantially parallel to the waveguide structure 14 and to the probe beam 12. As example is illustrated in FIG. 4. The pump beam 18 should travel along a path that passes very close to the surface of the temperature sensitive part 22 of the waveguide structure 14. Gas molecules in the vicinity of the temperature sensitive part 22 are heated by absorption of the pump beam, and the increased temperature of the gas molecules affects, e.g., the evanescent field of the probe beam 12 traveling through the waveguide structure 14. Changes in the amplitude and/or phase of the probe beam 12 can be measured by the light sensor 20. As the pump beam 18 is directed in parallel to the temperature sensitive part 22 of the waveguide structure 14, the interaction volume where the investigated gas molecules are heated can be increased. Thus, in some instances, a higher sensitivity can be achieved.

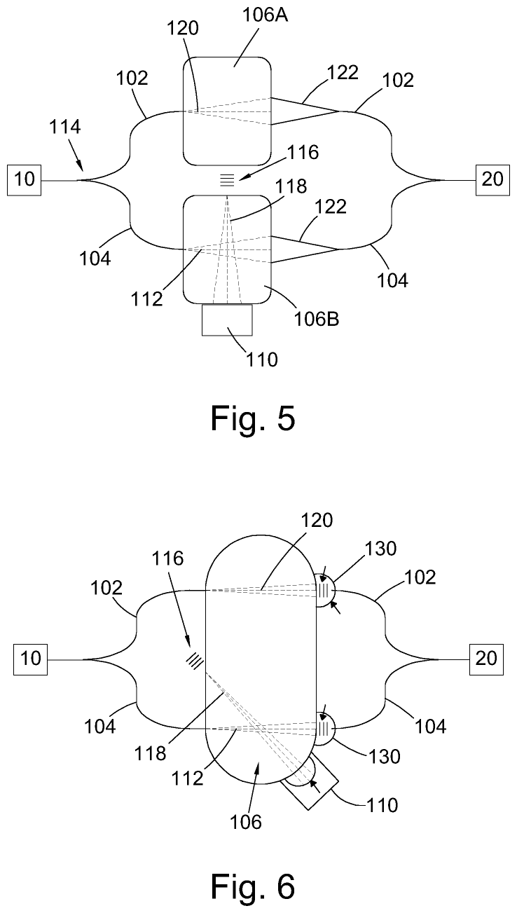

In some implementations, the integrated waveguide structure incorporates a Mach-Zehnder interferometer (MZI). Although FIG. 5 shows an example for a free space intersection between the probe and excitation beams, the MZI technique also can be used in arrangements like those of FIGS. 1, 2 and 4. As shown in the example of FIG. 5, the photo-thermal gas detection system includes an MZI having an integrated waveguide structure 114 that receives an optical beam from the light source 10. In this case, the light source 10 should produce coherent light. The integrated waveguide structure splits the optical beam into two beams and provides the beams, respectively, to parallel waveguides that define, respectively, a reference arm 102 and a probe arm 104. Each of the reference arm 102 and a probe arm 104 is interrupted by a respective channel 106A, 106B for the flow of the gas being investigated. The channels 106A, 106B can be formed, for example, as through silicon vias (TSVs) in the substrate on which the MZI waveguides structure is formed. In the illustrated example, the active area is in channel 106B, where the excitation (or pulse) beam crosses the probe beam 112 and is absorbed by a photodetector 110. The photodetector 110 is operable to generate a signal that can be used to provide feedback to control or tune the excitation light source 116, which can include, for example, a grating coupler fabricated on the surface of the substrate separating the channels 106A, 106B from one another. The grating coupler is operable to collect light from an excitation laser (e.g., VCSEL) or from an optical fiber or other light guide and to direct the collected light toward the area where the excitation and probe beams 112, 118 intersect. The reference light beam 120 travels through the reference arm 102 and passes through the channel 106A.

In operation, gas flows through both channels 106A, 106B. The through-hole 106B in interrupting the probe arm 104 enables gas to flow through the probe beam 112, and the pump beam 118 heats up the gas molecules through which the probe beam 112 passes. The refractive index of the air in the channel 106B changes as a result of the gas molecules heating up. The change in refractive index, in turn, affects the amplitude and/or phase. Light-collecting elements 122 are placed at the far end of the channels 106A, 106B to collect, respectively, the probe light beam 112 and the reference light beam 120 and to guide them back into the respective integrated waveguides 104, 102. The light collecting elements 122 can be implemented, for example, as inverted tapers, photonic crystals or plane lenses. The two arms 102, 104 of the waveguide structure merge the probe and reference light beams 112, 120 so as to create an interference pattern as the MZI output, which is coupled to the light detector 20. An ECU can receive signals from the light detector and can analyze the signals to recognize the presence of the gas molecules, to identify the particular gas molecule type, and to determine the gas concentration based on the detector output signals.

In some instances, instead of two separate TSVs 106A, 106B for the waveguide arms 102, 104 as in FIG. 5, a single TSV 106 in the substrate can serve as the channel for the interaction between the gas flow and the probe beam 112 as well as the reference beam 120, as illustrated in FIG. 6. Each of the reference arm 102 and the probe arm 104 of the waveguide structure can include a respective lens or reflecting cap 130, which may be composed, for example, of a metal hemisphere and which redirects the associated light beam 112 or 120 downward toward the surface. The configuration of FIG. 6 can, in some instances, result in a more equal gas flow in the probe and reference areas, and can decrease measurement distortion. Further, whereas the arrangement of FIG. 5 represents in-plane coupling of light passing through the channels 106A, 106B, the arrangement of FIG. 6 represents coupling of the same light by grating couplers.

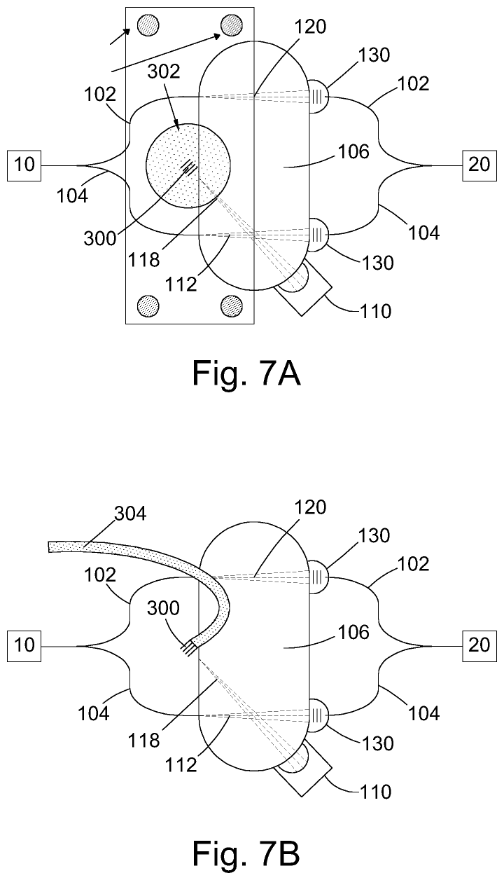

The photodetector 110 is operable to generate a signal that can be used to provide feedback to control or tune the excitation light source 116, which as described in connection with FIG. 5, can include, for example, a grating coupler fabricated on the surface of the substrate (e.g., substrate 24). As shown in FIGS. 7A and 7B, the grating coupler 300 is operable to collect light from an excitation laser (e.g., VCSEL) 302 (FIG. 7A) or from an optical fiber 304 (FIG. 7B), and to direct the collected light toward the area where the excitation (or pump) and probe beams 112, 118 intersect.

The photo-thermal gas detection systems described in this disclosure can be used, for example, in various modes of operation, depending on the application. In some cases, the excitation light surce is operable in a continuous mode, whereas in other situations, it is operable in a pulsed mode. For example, in implementations where the excited gas molecules impact the evanescent field of the probe beam, a pulsed mode of operation may be appropriate. Using pulsed excitation light can be useful, for example, for lock-in detection techniques.

Various aspects of the subject matter and the functional operations described in this specification (e.g., those of the ECU) can be implemented in digital electronic circuitry, or in computer software, firmware, or hardware, including the structures disclosed in this specification and their structural equivalents, or in combinations of one or more of them. Thus, aspects of the subject matter described in this specification can be implemented as one or more computer program products, i.e., one or more modules of computer program instructions encoded on a computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The apparatus can include, in addition to hardware, code that creates an execution environment for the computer program in question, e.g., code that constitutes processor firmware.

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

While this document contains many specific implementation details, these should not be construed as limitations on the scope of any inventions or of what may be claimed, but rather as descriptions of features specific to particular embodiments of particular inventions. Certain features that are described in this document in the context of separate embodiments can also can be implemented in combination in a single embodiment. Conversely, various features that are described in the context of a single embodiment can also can be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Further, various modifications will be readily apparent. Accordingly, other implementations are within the scope of the claims.

1. An apparatus comprising:
an integrated waveguide structure;
a first light source operable to produce a probe beam having a first wavelength, wherein the probe beam is coupled into a first end of the integrated waveguide structure;
a second light source operable to produce an excitation beam having a second wavelength to excite gas molecules in close proximity to a path of the probe beam; and
a light detector coupled to a second end of the integrated waveguide structure and operable to detect the probe beam after it passes through the integrated waveguide structure,
wherein the apparatus is operable such that excitation of the gas molecules results in a temperature increase of the gas molecules that induces a change in the probe beam that is measurable by the light detector.

2. The apparatus of claim 1 wherein the integrated waveguide structure includes a strip waveguide or a rib wave guide.

3. The apparatus of claim 1 wherein the integrated waveguide structure includes a temperature sensitive part where a path of the excitation beam intersects the path of the probe beam, wherein a change in temperature of the temperature sensitive part induces a change in the probe beam that is measurable by the light detector.

4. The apparatus of claim 1 wherein a path of the excitation beam intersects the path of the probe beam.

5. The apparatus of claim 1 comprising an electronic or optical feedback system to control or to tune the excitation beam.

6. The apparatus of claim 1 arranged such that a path of the excitation beam follows in close proximity to the path of the probe beam through the integrated waveguide structure.

7. The apparatus of claim 6 wherein the path of the excitation beam passes a portion of the integrated waveguide structure.

8. The apparatus of claim 1 wherein a path of the excitation beam intersects the path of the probe beam during free space propagation of the probe beam.

9. The apparatus of claim 1 wherein the integrated waveguide structure includes a Fabry-Perot interferometer.

10. The apparatus of claim 1 wherein the integrated waveguide structure includes a photonic crystal.

11. The apparatus of claim 1 wherein the integrated waveguide structure includes a Mach Zehnder interferometer.

12. The apparatus of claim 11 wherein the integrated waveguide structure has a reference arm and a probe arm.

13. The apparatus of claim 12 having at least one opening in a substrate on which the integrated waveguide structure is disposed, the at least one opening enabling flow of a gas at a location where the excitation beam intersects the probe beam.

14. The apparatus of claim 13 wherein having a plurality of openings in the substrate, wherein the apparatus is operable such that a measurement portion of the probe beam travels through a first one of the openings, and a reference portion of the probe beam travels through a second one of the openings.

15. The apparatus of claim 1 wherein the second light source is operable in a pulsed mode.

16. The apparatus of claim 1 wherein the second light source is operable in a continuous mode.

17. The apparatus of claim 1 further including an optical element operable to direct the excitation beam toward an area where the excitation and probe beams intersect.

18. The apparatus of claim 1 further including a light guide to guide the excitation beam from the second light source to a grating coupler, wherein the grating coupler is operable to direct the excitation beam toward an area where the excitation and probe beams intersect.

19. The apparatus of claim 1 wherein the excitation beam has a wavelength different from the wavelength of the probe beam.

20. A method comprising:
producing a probe beam having a first wavelength;
coupling the probe beam into a first end of an integrated waveguide structure;
producing an excitation beam having a second wavelength to excite gas molecules in close proximity to a path of the probe beam, wherein excitation of the gas molecules results in a temperature increase of the gas molecules that induces a change in the probe beam; and
measuring, by a light detector coupled to a second end of the integrated waveguide structure, the change in the probe beam.
